(19) United States
(12) Patent Application Publication
(10) Pub. No.: US 2024/0012964 A1
(43) Pub. Date: Jan. 11, 2024

(54) CLOSED LOOP SIMULATION PLATFORM FOR ACCELERATED POLYMER ELECTROLYTE MATERIAL DISCOVERY

(71) Applicant: TOYOTA RESEARCH INSTITUTE, INC., Los Altos, CA (US)

(72) Inventors: Daniel SCHWEIGERT, Richmond, CA (US); Ha-Kyung KWON, New York, NY (US); Arash KHAJEH, Merced, CA (US)

(73) Assignees: TOYOTA RESEARCH INSTITUTE, INC., Los Altos, CA (US); TOYOTA JIDOSHA KABUSHIKI KAISHA, Aichi-Ken (JP)

(21) Appl. No.: 17/860,016

(22) Filed: Jul. 7, 2022

(51) Int. Cl. G06F 30/27

(52) U.S. Cl. CPC G06F 30/27

(57) ABSTRACT

A method of closed loop simulation for accelerated material discovery is described. The method includes ranking a plurality of candidate systems according to corresponding properties of interest predicted by a first prediction model. The method also includes simulating a first top-N of the plurality of candidate systems according to the corresponding properties of interest predicted by the first prediction model. The method further includes re-ranking the plurality of candidate systems according to the corresponding properties of interest predicted by a second prediction model. The method also includes simulating a second top-N of the plurality of candidate systems according to the corresponding properties of interest predicted by the second prediction model.

### TECHNICAL FIELD

Certain aspects of the present disclosure generally relate to artificial neural networks and, more particularly, to a closed loop simulation platform for accelerated polymer electrolyte material discovery.

### BACKGROUND

An artificial neural network, which may include an interconnected group of artificial neurons, may be a computational device or may represent a method to be performed by a computational device. Artificial neural networks may have corresponding structure and/or function in biological neural networks. Artificial neural networks, however, may provide useful computational techniques for certain applications, in which traditional computational techniques may be cumbersome, impractical, or inadequate. Because artificial neural networks may infer a function from observations, such networks may be useful in applications where the complexity of the task and/or data makes the design of the function burdensome using conventional techniques.

Machine learning may be used to perform both materials discovery and predict properties of the materials faster than molecular simulations. Machine learning can help identify correlations between material features and target properties. Nevertheless, it is challenging to accelerate the computational search for a suitable polymer electrolyte material that can be used in batteries. A method to accelerate the computational search for a suitable polymer electrolyte material that can be used in batteries is desired.

### SUMMARY

A method of closed loop simulation for accelerated material discovery is described. The method includes ranking a plurality of candidate systems according to corresponding properties of interest predicted by a first prediction model. The method also includes simulating a first top-N of the plurality of candidate systems according to the corresponding properties of interest predicted by the first prediction model. The method further includes re-ranking the plurality of candidate systems according to the corresponding properties of interest predicted by a second prediction model. The method also includes simulating a second top-N of the plurality of candidate systems according to the corresponding properties of interest predicted by the second prediction model.

A non-transitory computer-readable medium having program code recorded thereon for compositional feature representation learning of closed loop simulation for accelerated material discovery is described. The program code being is by a processor. The non-transitory computer-readable medium includes program code to rank a plurality of candidate systems according to corresponding properties of interest predicted by a first prediction model. The non-transitory computer-readable medium also includes program code to simulate a first top-N of the plurality of candidate systems according to the corresponding properties of interest predicted by the first prediction model. The non-transitory computer-readable medium further includes program code to re-rank the plurality of candidate systems according to the corresponding properties of interest predicted by a second prediction model. The non-transitory computer-readable medium also includes program code to simulate a second top-N of the plurality of candidate systems according to the corresponding properties of interest predicted by the second prediction model.

A system for compositional feature representation learning of closed loop simulation for accelerated material discovery is described. The system includes a first prediction model to rank a plurality of candidate systems according to corresponding properties of interest predicted by the first prediction model. The system also includes a molecular dynamics simulation cluster to simulate a first top-N of the plurality of candidate systems according to the corresponding properties of interest predicted by the first prediction model. The system further includes a second prediction model to re-rank the plurality of candidate systems according to the corresponding properties of interest predicted by the second prediction model. The system also includes the molecular dynamics simulation cluster to simulate a second top-N of the plurality of candidate systems according to the corresponding properties of interest predicted by the second prediction model.

This has outlined, rather broadly, the features and technical advantages of the present disclosure in order that the detailed description that follows may be better understood. Additional features and advantages of the disclosure will be described below. It should be appreciated by those skilled in the art that this disclosure may be readily utilized as a basis for modifying or designing other structures for carrying out the same purposes of the present disclosure. It should also be realized by those skilled in the art that such equivalent constructions do not depart from the teachings of the disclosure as set forth in the appended claims. The novel features, which are believed to be characteristic of the disclosure, both as to its organization and method of operation, together with further objects and advantages, will be better understood from the following description when considered in connection with the accompanying figures. It is to be expressly understood, however, that each of the figures is provided for the purpose of illustration and description only and is not intended as a definition of the limits of the present disclosure.

### BRIEF DESCRIPTION OF THE DRAWINGS

The features, nature, and advantages of the present disclosure will become more apparent from the detailed description set forth below when taken in conjunction with the drawings in which like reference characters identify correspondingly throughout.

FIG. 1 illustrates an example implementation of designing a neural network using a system-on-chip (SoC), including a general purpose processor, in accordance with certain aspects of the present disclosure.

FIGS. 2A, 2B, and 2C are diagrams illustrating a neural network, in accordance with aspects of the present disclosure.

FIG. 2D is a diagram illustrating a neural network, in accordance with aspects of the present disclosure.

FIG. 3 is a block diagram illustrating an overview of a closed loop simulation platform for accelerated polymer electrolyte material discovery, in accordance with aspects of the present disclosure.

FIG. 4 is a flow diagram illustrating a method of closed loop simulation for accelerated material discovery, according to aspects of the present disclosure.

### DETAILED DESCRIPTION

The detailed description set forth below, in connection with the appended drawings, is intended as a description of various configurations and is not intended to represent the only configurations in which the concepts described may be practiced. The detailed description includes specific details for the purpose of providing a thorough understanding of the various concepts. Nevertheless, it will be apparent to those skilled in the art that these concepts may be practiced without these specific details. In some instances, well-known structures and components are shown in block diagram form in order to avoid obscuring such concepts.

Based on the teachings, one skilled in the art should appreciate that the scope of the disclosure is intended to cover any aspect of the disclosure, whether implemented independently of or combined with any other aspect of the disclosure. For example, an apparatus may be implemented or a method may be practiced using any number of the aspects set forth. In addition, the scope of the disclosure is intended to cover such an apparatus or method practiced using other structure, functionality, or structure and functionality in addition to or other than the various aspects of the disclosure set forth. It should be understood that any aspect of the disclosure disclosed may be embodied by one or more elements of a claim.

Although particular aspects are described, many variations and permutations of these aspects fall within the scope of the disclosure. Although some benefits and advantages of the preferred aspects are mentioned, the scope of the disclosure is not intended to be limited to particular benefits, uses, or objectives. Rather, aspects of the disclosure are intended to be broadly applicable to different technologies, system configurations, networks, and protocols, some of which are illustrated by way of example in the figures and in the following description of the preferred aspects. The detailed description and drawings are merely illustrative of the disclosure, rather than limiting the scope of the disclosure being defined by the appended claims and equivalents thereof.

Machine learning is concerned with the automatic discovery of patterns in data through the use of computer algorithms. Once discovered, these patterns may be used to perform data classification and/or value prediction. With growing experimental and simulated dataset size for materials science research, the ability of algorithms to automatically learn and improve from data becomes increasingly useful. Various types of machine learning algorithms, such as neural networks, have recently been applied to materials research. Among these machine learning algorithms, convolutional neural networks (CNNs) have been very attractive in recent years due to their great success in image recognition.

A CNN may be composed of multilayer neural networks, of which at least one layer employs a mathematical operation called “convolution” to enable the CNN to extract high-level features directly from data. Compared to many other algorithms that specify artificial features based on domain knowledge, a CNN involves relatively little pre-processing, as the features can be directly learned from the data. This is particularly useful when the features are difficult to exactly define. Unlike their long-used basic forms, such as perceptron and fully connected neural networks, CNNs are very recently used for solving solid state problems, such as learning material property prediction, material classification, and material phase transition identification.

Another advantage of neural networks is that they are easy to utilize in transfer learning, which means that a neural network first learns from a large database with inexpensive labels (e.g., first principles calculation results), and then it is fine-tuned on a small dataset where much fewer labeled samples are available (e.g., experimental data). This technique can be used to overcome the data scarcity problem in materials research, and it is applied to property prediction of small molecules and crystalline compounds very recently as a tool for accelerated materials discovery.

The practical realization and sustainable future of emergent technologies is dependent on accelerating materials discovery. Data-driven methods are anticipated to play an increasingly significant role in enabling this desired acceleration. While the vision of accelerating materials discovery using data-driven methods is well-founded, practical realization is throttled due to challenges in data generation, ingestion, and materials state-aware machine learning. High-throughput experiments and automated computational workflows are addressing the challenge of data generation, and capitalizing on these emerging data resources involves ingestion of data into an architecture that captures the complex provenance of experiments and simulations.

Traditional computational search for polymer electrolyte materials involves molecular dynamics simulations. As described, molecular dynamics simulations involve computation of the macroscopic properties of a material from the processes at the atomistic level that are the direct result of the simulation. For accurate results, the simulations are carried out for a long period of time and, therefore, are computationally expensive. The long computation time and the vast number of material systems that are potentially screened by this method render the full computational search for next generation polymer electrolyte materials unrealistic due to the multiplying computational costs involved.

In practice, machine learning may be used to perform both materials discovery and predict properties of the materials faster than molecular simulations. Machine learning can help identify correlations between material features and target properties. Machine learning may be used to perform both materials discovery and predict properties of the materials faster than molecular simulations. Machine learning can help identify correlations between material features and target properties. Nevertheless, it is challenging to accelerate the computational search for a suitable polymer electrolyte material that can be used in batteries. A method to accelerate the computational search for a suitable polymer electrolyte material that can be used in batteries is desired.

Some aspects of the present disclosure are directed to a closed loop simulation platform to accelerate the computational search for a suitable polymer electrolyte material that can be used in batteries. In these aspects of the present disclosure, the closed loop simulation platform can reduce the computational cost involved in the search for polymer electrolytes with improved performance. In one aspect of the present disclosure, the closed loop simulation platform operates by applying prediction models at various stages in the process of simulating candidate systems and autonomously reprioritizing and reassigning computer resources for the candidate system simulations based on the prediction model results. As described, the term “candidate system” may refer to materials (e.g., polymers and salts), the concentration of these materials (e.g., polymers and salts), as well as the temperature and pressure at which simulations are run. Based on this definition, two candidate systems with the exact same materials may have varying ranking, because one concentration of the candidate system might lead to a higher conductivity than another candidate system.

Some aspects of the present disclosure ensure that the prediction models stay up-to-date with knowledge gained from computational simulation results. These aspects of the present disclosure involve re-training the prediction models as new result data becomes available. That way, prediction models used to reprioritize simulation efforts are always informed by the latest ground-truth data available. The constantly recurring reprioritization of candidate systems for assigning computational resources during simulation accelerates the rate of discovery of new high performance materials over conventional computational screening. For example, the prediction models are not just choosing the next polymer material to rank (and subsequently simulate), but are selecting the polymer(s), the salt(s), the concentration of each component, and the temperature of the system.

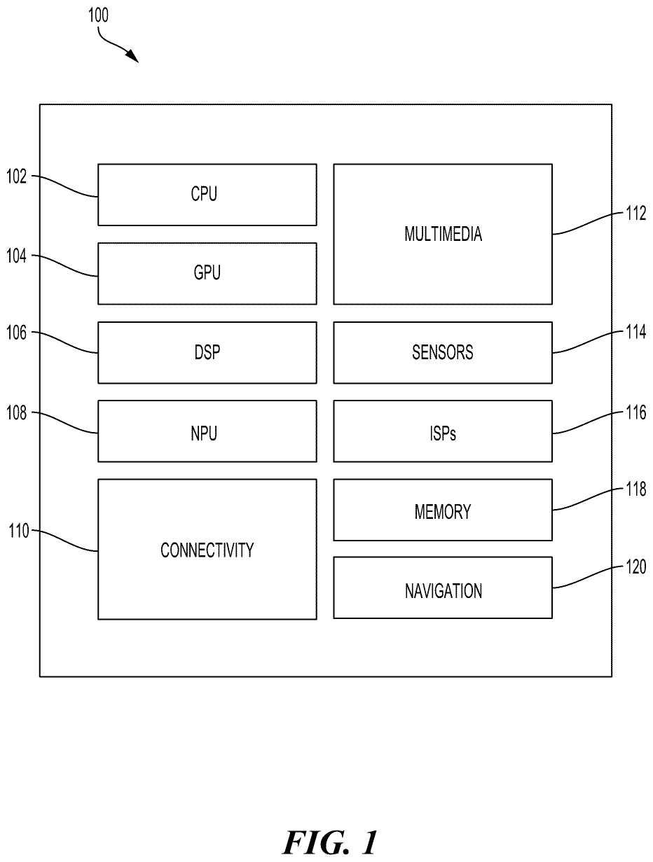

FIG. 1 illustrates an example implementation of a system-on-chip (SoC) 100, which may include a central processing unit (CPU) 102 or multi-core CPUs, in accordance with certain aspects of the present disclosure, such an artificial intelligence (AI) material state prediction. Variables (e.g., neural signals and synaptic weights), system parameters associated with a computational device (e.g., neural network with weights), delays, frequency bin information, and task information may be stored in a memory block associated with a neural processing unit (NPU) 108, in a memory block associated with a CPU 102, in a memory block associated with a graphics processing unit (GPU) 104, in a memory block associated with a digital signal processor (DSP) 106, in a memory block 118, or may be distributed across multiple blocks. Instructions executed at the CPU 102 may be loaded from a program memory associated with the CPU 102 or may be loaded from a memory block 118.

The SoC 100 may also include additional processing blocks tailored to specific functions, such as a connectivity block 110, which may include fifth generation (5G) new radio (NR) connectivity, fourth generation long term evolution (4G LTE) connectivity, unlicensed Wi-Fi connectivity, USB connectivity, Bluetooth connectivity, and the like, and a multimedia processor 112 that may, for example, detect and recognize gestures. In one implementation, the NPU is implemented in the CPU, DSP, and/or GPU. The SoC 100 may also include a sensor processor 114 to provide sensor image data, image signal processors (ISPs) 116, and/or navigation module 120, which may include a global positioning system.

Deep learning architectures may perform an object recognition task by learning to represent inputs at successively higher levels of abstraction in each layer, thereby building up a useful feature representation of the input data. In this way, deep learning addresses a major bottleneck of traditional machine learning. Prior to the advent of deep learning, a machine learning approach to an object recognition problem may have relied heavily on human engineered features, perhaps in combination with a shallow classifier. A shallow classifier may be a two-class linear classifier, for example, in which a weighted sum of the feature vector components may be compared with a threshold to predict to which class the input belongs. Human engineered features may be templates or kernels tailored to a specific problem domain by engineers with domain expertise. Deep learning architectures, in contrast, may learn to represent features that are similar to what a human engineer might design, but through training. Furthermore, a deep network may learn to represent and recognize new types of features that a human might not have considered.

A deep learning architecture may learn a hierarchy of features. If presented with visual data, for example, the first layer may learn to recognize relatively simple features, such as edges, in the input stream. In another example, if presented with auditory data, the first layer may learn to recognize spectral power in specific frequencies. The second layer, taking the output of the first layer as input, may learn to recognize combinations of features, such as simple shapes for visual data or combinations of sounds for auditory data. For instance, higher layers may learn to represent complex shapes in visual data or words in auditory data. Still higher layers may learn to recognize common visual objects or spoken phrases.

Deep learning architectures may perform especially well when applied to problems that have a natural hierarchical structure. For example, the classification of motorized vehicles may benefit from first learning to recognize wheels, windshields, and other features. These features may be combined at higher layers in different ways to recognize cars, trucks, and airplanes.

Neural networks may be designed with a variety of connectivity patterns. In feed-forward networks, information is passed from lower to higher layers, with each neuron in a given layer communicating to neurons in higher layers. A hierarchical representation may be built up in successive layers of a feed-forward network, as described above. Neural networks may also have recurrent or feedback (also called top-down) connections. In a recurrent connection, the output from a neuron in a given layer may be communicated to another neuron in the same layer. A recurrent architecture may be helpful in recognizing patterns that span more than one of the input data chunks that are delivered to the neural network in a sequence. A connection from a neuron in a given layer to a neuron in a lower layer is called a feedback (or top-down) connection. A network with many feedback connections may be helpful when the recognition of a high-level concept may aid in discriminating the particular low-level features of an input.

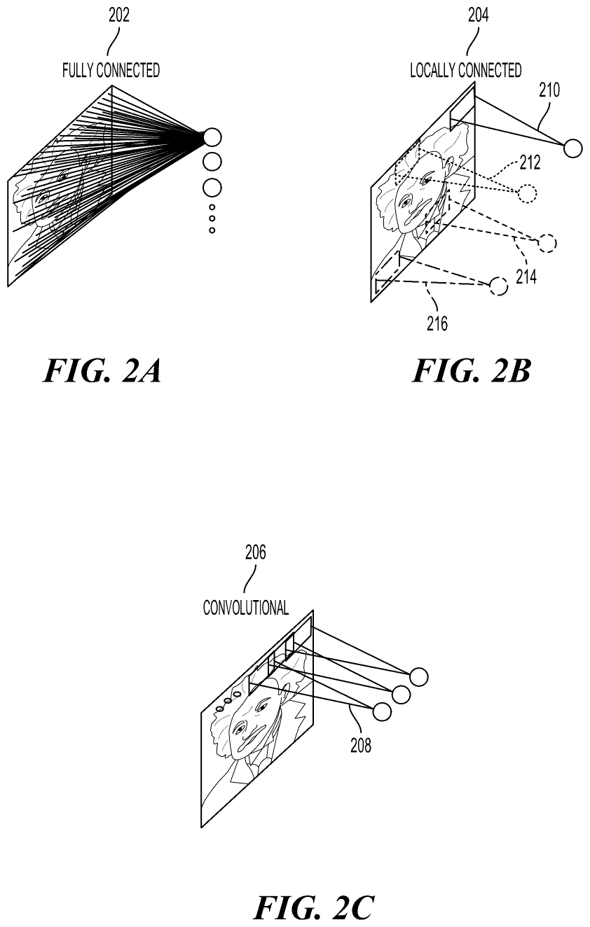

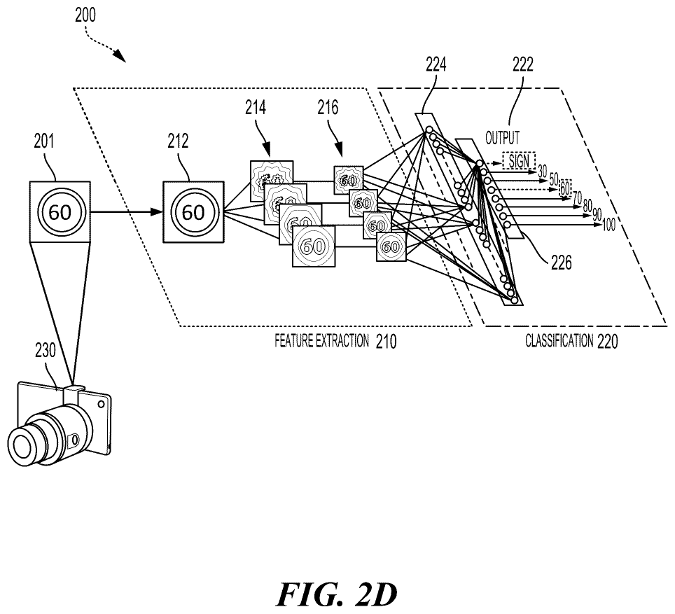

FIGS. 2A, 2B, and 2C are diagrams illustrating a neural network, in accordance with aspects of the present disclosure. The connections between layers of the neural network shown in FIG. 2A-2C may be fully connected or locally connected. FIG. 2A illustrates an example of a fully connected neural network 202. In a fully connected neural network 202, a neuron in a first layer may communicate its output to every neuron in a second layer, so that each neuron in the second layer will receive input from every neuron in the first layer.

FIG. 2B illustrates an example of a locally connected neural network 204. In a locally connected neural network 204, a neuron in a first layer may be connected to a limited number of neurons in the second layer. More generally, a locally connected layer of the locally connected neural network 204 may be configured so that each neuron in a layer will have the same or a similar connectivity pattern, but with connection strengths that may have different values (e.g., 210, 212, 214, and 216). The locally connected connectivity pattern may give rise to spatially distinct receptive fields in a higher layer, because the higher layer neurons in a given region may receive inputs that are tuned through training to the properties of a restricted portion of the total input to the network.

FIG. 2C illustrates an example of a locally connected neural network is a convolutional neural network. As shown in FIG. 2C, an example of a locally connected neural network is provided as a convolutional neural network 206. The convolutional neural network 206 may be configured such that the connection strengths associated with the inputs for each neuron in the second layer are shared (e.g., 208). Convolutional neural networks may be well suited to problems in which the spatial location of inputs is meaningful.

FIG. 2D illustrates one type of convolutional neural network, referred to as a deep convolutional network (DCN). In particular, FIG. 2D illustrates a detailed example of a DCN 200 designed to recognize visual features from an image 201, which is provided as an input from an image capturing device 230, such as a car-mounted camera. The DCN 200 of the current example may be trained to identify traffic signs and a number provided on the traffic sign. Of course, the DCN 200 may be trained for other tasks, such as identifying lane markings or identifying traffic lights, or predicting states of material sample following processing.

The DCN 200 may be trained with supervised learning. During training, the DCN 200 may be presented with an image, such as the image 201 of a speed limit sign, and a forward pass may then be computed to produce an output 222. The DCN 200 may include a feature extraction section 210 and a classification section 220. Upon receiving the image 201, a convolutional layer 212 may apply convolutional kernels (not shown) to the image 201 to generate a first set of feature maps 214. As an example, the convolutional kernel for the convolutional layer 212 may be a 5×5 kernel that generates 28×28 feature maps. In the present example, because four different convolutional kernels were applied to the image 201 at the convolutional layer 212, four different feature maps are generated in the first set of feature maps 214. The convolutional kernels may also be referred to as filters or convolutional filters.

The first set of feature maps 214 may be subsampled by a max pooling layer (not shown) to generate a second set of feature maps 216. The max pooling layer reduces the size of the first set of feature maps 214. That is, a size of the second set of feature maps 216, such as 14×14, is less than the size of the first set of feature maps 214, such as 28×28. The reduced size provides similar information to a subsequent layer while reducing memory consumption. The second set of feature maps 216 may be further convolved via one or more subsequent convolutional layers (not shown) to generate one or more subsequent sets of feature maps (not shown).

In the example of FIG. 2D, the second set of feature maps 216 is convolved to generate a first feature vector 224. Furthermore, the first feature vector 224 is further convolved to generate a second feature vector 226. Each feature of the second feature vector 226 may include a number that corresponds to a possible feature of the image 201, such as “sign,” “60,” and “100.” A softmax function (not shown) may convert the numbers in the second feature vector 226 to a probability. As such, an output 222 of the DCN 200 is a probability of the image 201 including one or more features.

In the present example, the probabilities in the output 222 for “sign” and “60” are higher than the probabilities of the others of the output 222, such as “30,” “40,” “50,” “70,” “80,” “90,” and “100.” Before training, the output 222 produced by the DCN 200 is likely to be incorrect. Thus, an error may be calculated between the output 222 and a target output. The target output is the ground truth of the image 201 (e.g., “sign” and “60”). The weights of the DCN 200 may then be adjusted so the output 222 of the DCN 200 is more closely aligned with the target output.

As shown in FIGS. 2A-2D, machine learning is concerned with the automatic discovery of patterns in data through the use of computer algorithms. Once discovered, these patterns may be used to perform data classification and/or value prediction. With growing experimental and simulated dataset size for materials science research, the ability of algorithms to automatically learn and improve from data becomes increasingly useful. Various types of machine learning algorithms, such as neural networks, have recently been applied to materials research. Among these machine learning algorithms, convolutional neural networks (CNNs) have been very attractive in recent years due to their great success in image recognition.

The DCN 200 shown in FIG. 2D is composed of multilayer neural networks, of which at least one layer employs a mathematical operation called “convolution” to enable the DCN 200 to extract high-level features directly from data. Compared to many other algorithms that specify artificial features based on domain knowledge, the DCN 200 involves relatively little pre-processing, as the features can be directly learned from the data, such as the image 201. This is particularly useful when the features are difficult to exactly define. Unlike their long-used basic forms, such as perceptron and fully connected neural networks, DCNs are very recently used for solving solid state problems, such as learning material property prediction, material classification, and material phase transition identification.

Another advantage of neural networks is that they are easy to utilize in transfer learning, which means that a neural network first learns from a large database with inexpensive labels (e.g., first principles calculation results), and then it is fine-tuned on a small dataset where much fewer labeled samples are available (e.g., experimental data). This technique can be used to overcome the data scarcity problem in materials research, and it is applied to property prediction of small molecules and crystalline compounds very recently as a tool for accelerated materials discovery.

The practical realization and sustainable future of emergent technologies is dependent on accelerating materials discovery using neural networks. Data-driven methods are anticipated to play an increasingly significant role in enabling this desired acceleration. While the vision of accelerating materials discovery using data-driven methods is well-founded, practical realization is throttled due to challenges in data generation, ingestion, and materials state-aware machine learning. High-throughput experiments and automated computational workflows are addressing the challenge of data generation, and capitalizing on these emerging data resources involves ingestion of data into an architecture that captures the complex provenance of experiments and simulations.

Traditional computational search for polymer electrolyte materials involves molecular dynamics simulations. As described, molecular dynamics simulations involve computation of the macroscopic properties of a material from the processes at the atomistic level that are the direct result of the simulation. For accurate results, the simulations are carried out for a long period of time and, therefore, are computationally expensive. The long computation time and the vast number of material systems that are potentially screened by this method render the full computational search for next generation polymer electrolyte materials unrealistic due to the multiplying computational costs involved.

In practice, machine learning may be used to perform both materials discovery and predict properties of the materials faster than molecular simulations. Machine learning can help identify correlations between material features and target properties. Machine learning may be used to perform both materials discovery and predict properties of the materials faster than molecular simulations. Machine learning can help identify correlations between material features and target properties. Nevertheless, it is challenging to accelerate the computational search for a suitable polymer electrolyte material that can be used in batteries. A method to accelerate the computational search for a suitable polymer electrolyte material that can be used in batteries is desired.

Some aspects of the present disclosure are directed to a closed loop simulation platform to accelerate the computational search for a suitable polymer electrolyte material that can be used in batteries. In these aspects of the present disclosure, the closed loop simulation platform can reduce the computational cost involved in the search for polymer electrolytes with improved performance. In one aspect of the present disclosure, the closed loop simulation platform operates by applying prediction models at various stages in the process of simulating candidate systems and autonomously reprioritizing and reassigning computer resources for the candidate system simulations based on the prediction model results. As described, the term “candidate system” may refer to materials (e.g., polymers and salts), the concentration of these materials, as well as the temperature and pressure at which simulations are run). Based on this definition, two candidate systems with the exact same materials may have varying ranking, because one concentration of the candidate system might lead to a higher conductivity than the candidate system.

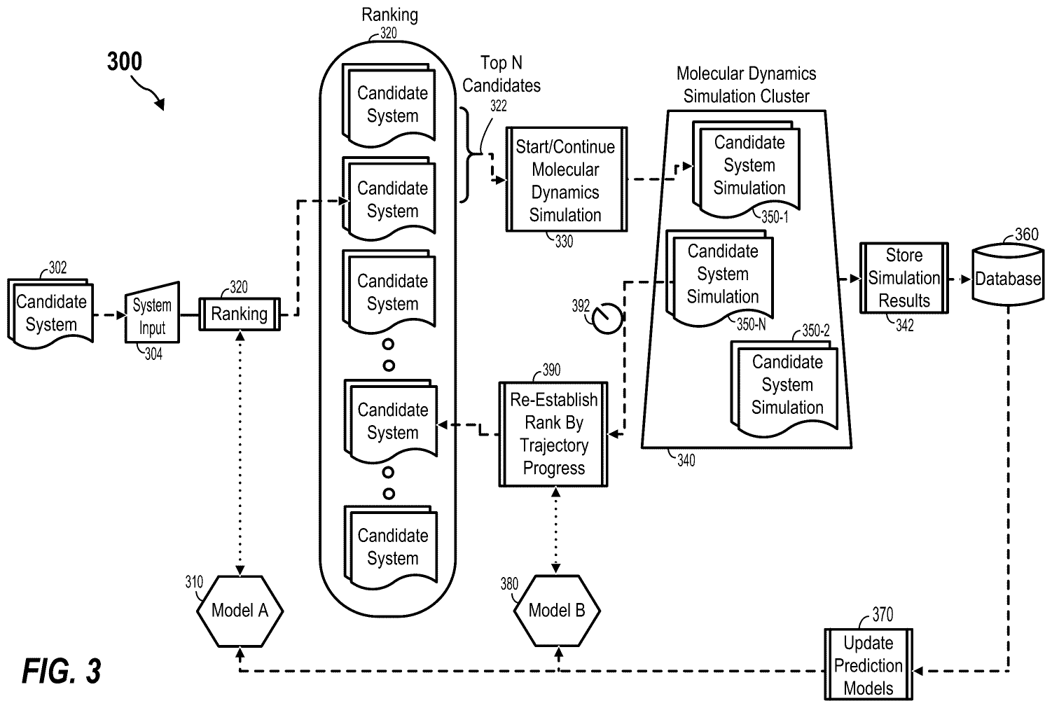

Some aspects of the present disclosure ensure that the prediction models stay up-to-date with knowledge gained from computational simulation results. These aspects of the present disclosure involve re-training the prediction models as new result data becomes available. That way, prediction models used to reprioritize simulation efforts are always informed by the latest ground-truth data available. The constantly recurring reprioritization of candidate systems for assigning computational resources during simulation accelerates the rate of discovery of new high performance materials over conventional computational screening, for example, as shown in FIG. 3.

FIG. 3 is a block diagram illustrating an overview of a closed loop simulation platform for accelerated polymer electrolyte material discovery, in accordance with aspects of the present disclosure. As shown in FIG. 3, the closed loop simulation platform 300 includes a molecular dynamics simulation cluster 340, capable of running parallelized molecular dynamics simulations. The molecular dynamics simulation cluster 340 may be implemented using an open source large-scale atomic/molecular massively parallel simulator (LAMMPS) framework. In this configuration, the molecular dynamics simulation cluster 340 periodically checks, at block 392, if any of the candidate system simulations (e.g., 350-1, . . . , 350-N) currently running in the molecular dynamics simulation cluster 340 have run sufficiently long to invoke a second prediction model 380. In some aspects of the present disclosure, the respective prediction results from the second prediction model 380 are used to update a ranking procedure 320 of candidate systems. Based on the ranking procedure 320, the closed loop simulation platform 300, at block 330, starts/stops/resumes the candidate system simulations 350 in the molecular dynamics simulation cluster 340.

In some aspects of the present disclosure, the periodic check performed by the molecular dynamics simulation cluster 340 to start/continue at block 330 and stop, the candidate system simulations 350 is based on a predetermined parameter. At block 342, the molecular dynamics simulation cluster 340 stores simulation results in a database 360, or other like designated location (e.g., a cloud data warehouse or data lake). In some aspects of the present disclosure, the immediate results of the simulation are collections of coordinates of individual atoms involved in the simulation and their evolution in time.

As described, dataset collections of coordinates of individual atoms involved in simulation and their evolution in time are referred to as trajectories. In practice, state-of-the-art and community-known algorithmic methods are applied to completed trajectories in order to compute macroscopic material properties of interest. Primarily, these properties are the conductivity and the diffusivity of lithium (Li+) ions in the simulated material. A material with a high diffusivity and conductivity in comparison with state-of-the-art materials has a potential to outperform existing polymer electrolytes in energy storage applications when realized in the physical world. Some aspects of the present disclosure are directed to address the simulation aspects, however, and are not concerned with the physical realization of these materials.

In practice, the number of conceptualized materials that could potentially undergo such computational simulation using the molecular dynamics simulation cluster 340 is virtually unlimited. In some aspects of the present disclosure, the closed loop simulation platform 300 applies a first prediction model 310 and a second prediction model 380 to predict property estimates for a diffusivity property and a conductivity property by which a candidate system 302, after a system input 304, can be ranked in priority, according to a ranking procedure 320. Along with the predicted estimates, the first prediction model 310 and the second prediction model 380 provide a measure of prediction uncertainty, which is taken into account in the ranking procedure 320 of the material candidates. In some aspects of the present disclosure, the specific ranking procedure for determining the top-N candidates 322 is a function that takes into account property prediction estimates and prediction uncertainty for optimized decision making. The concrete implementation can start with simple heuristics (e.g., maximize properties while minimizing uncertainty) and is a matter of optimization and therefore may evolve over time.

In this example, the first prediction model 310 may predict a resulting lithium (Li+) ion conductivity as a simulation process and an estimate of the prediction uncertainty from the system input 304 of the candidate system 302. In some aspects of the present disclosure, the system input 304 provides a 2D representation of the candidate system 302, such as a simplified molecular-input line-entry system (SMILES) string. The SMILES string is a text representation that may be transformed into a numerical vector representation applying a set of featurization methods. These vector representations are fed into the prediction model to obtain the predicted quantities.

In some aspects of the present disclosure, the second prediction model 380 predicts a resulting lithium (Li+) ion conductivity as a simulation process and an estimate of the prediction uncertainty. In this example, the second prediction model 380 performs the predictions from an input of available trajectory data of a material's partially progressed simulation, for example, based on the candidate system simulations 350 (350-1, . . . , 350-N) of the molecular dynamics simulation cluster 340. The spatial-temporal data from the not yet completed simulation trajectory (e.g., a partially completed material candidate simulation) is fed into the second prediction model 380 to obtain the predicted quantities. In some aspects of the present disclosure, the predicted quantities are used to re-establish rank by trajectory process at block 390.

In aspects of the present disclosure, the second prediction model 380 can be applied at various stages of progress of the simulation by the molecular dynamics simulation cluster 340. The progress, as well as the interval and frequency at which the second prediction model 380 is applied, is a parameter of the entire system, which can be optimized for best performance to start/continue the molecular dynamics simulation at block 330 and stop the molecular dynamics simulation at block 392, for example, according to a predetermined parameter. Optionally, further prediction models can be applied as they become available to aid in the prioritization of material candidates at block 390.

Some specifications for the first prediction model 310 and the second prediction model 380 include an ability to featurize raw input (e.g., SMILES strings or trajectory data) into numerical inputs, which may be performed using the system input 304. Another specification for the first prediction model 310 and the second prediction model 380 is reasonable prediction quality for macroscopic properties of interest (e.g., conductivity, diffusivity, etc.). Another specification for the first prediction model 310 and the second prediction model 380 is the ability to estimate prediction uncertainty or other measures of the specific prediction quality. A further specification for the first prediction model 310 and the second prediction model 380 is trainability; namely, the ability to improve prediction quality and reduce prediction uncertainty by training with new data. Machine learning topologies which can provide this type of capability include, but are not limited to, Gaussian process repressors and/or neural networks.

In some aspects of the present disclosure, the first prediction model 310 and/or the second prediction model 380 are applied whenever applicable input data is available. The computational cost of applying the first prediction model 310 and/or the second prediction model 380 is very small relative to the computational costs involved in running the molecular dynamics simulation cluster 340. Therefore, the first prediction model 310 and/or the second prediction model 380 may be continuously applied to material candidates as a lightweight background process. In this example, the results of these predictions feed into a ranking procedure 320, which lists all candidate systems in a top-down ranking.

As computational resources for simulations are typically limited due to real-world constraints, such as server capacity and budgets, a user of the closed loop simulation platform 300 can decide to continuously run the molecular dynamics simulation cluster 340 for a fixed number of the top-N candidates 322. In this example, the closed loop simulation platform 300 chooses the top-N candidates 322 according to a priority ranking list from the ranking procedure 320 for simulations in the molecular dynamics simulation cluster 340. In some aspects of the present disclosure, the closed loop simulation platform 300 reacts in real-time to changes in the ranking procedure 320 by reassigning simulation resources among material candidates, which by circumstance can mean pausing ongoing simulations and starting or continuing other simulations at block 330.

As computational resources for simulations are typically limited due to real-world constraints, such as server capacity and budgets, a user of the closed loop simulation platform 300 can decide to continuously run the molecular dynamics simulation cluster 340 for a fixed number of the top-N candidates 322. In this example, the closed loop simulation platform 300 chooses the top-N candidates 322 according to a priority ranking list from the ranking procedure 320 for simulations in the molecular dynamics simulation cluster 340. In some aspects of the present disclosure, the closed loop simulation platform 300 reacts in real-time to changes in the ranking procedure 320 by reassigning simulation resources among material candidates, which by circumstance can mean pausing ongoing simulations and starting or continuing other simulations at block 330.

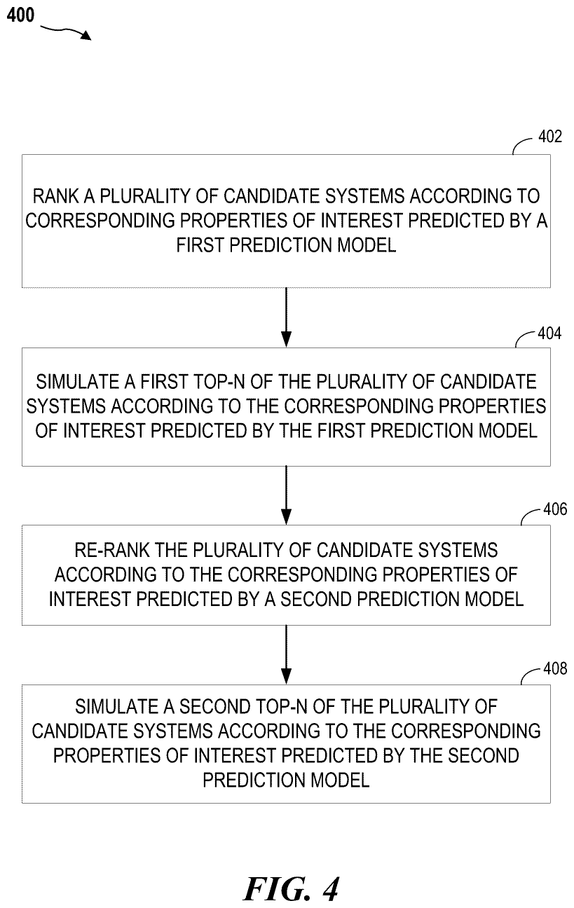

In some aspects of the present disclosure, the first prediction model 310 and/or the second prediction model 380 are updated as new completed simulation data is available. The first prediction model 310 and/or the second prediction model 380 are then re-run on available candidates which can lead to a reprioritization among the material candidates. As noted above, the first prediction model 310 and/or the second prediction model 380 are updated as new ground truth data becomes available. This process occurs in a fully automated manner and includes a rigorous process of cross validation to ensure generalizability of the models to the extent possible given by the available data. This process is further illustrated, for example, according to a method as shown in FIG. 4.

FIG. 4 is a flow diagram illustrating a method of closed loop simulation for accelerated material discovery, according to aspects of the present disclosure. A method 400 begins at block 402, in which a plurality of candidate systems are ranked according to corresponding properties of interest predicted by a first prediction model. For example, FIG. 3 shows the closed loop simulation platform 300 applying the first prediction model 310 to predict property estimates for a diffusivity property and a conductivity property by which the candidate system 302, after system input 304, can be ranked in priority, according to a ranking procedure 320. Along with the predicted estimates, the first prediction model 310 provides a measure of prediction uncertainty, which is taken into account in the ranking procedure 320 of the material candidates. In some aspects of the present disclosure, the ranking procedure 320, for determining the top-N candidates 322, is a function that takes into account property prediction estimates and prediction uncertainty for optimized decision making.

At block 404, a first top-N of the plurality of candidate systems is simulated according to the corresponding properties of interest predicted by the first prediction model. For example, in FIG. 3 the closed loop simulation platform 300 includes a molecular dynamics simulation cluster 340, capable of running parallelized molecular dynamics simulations. In this configuration, the molecular dynamics simulation cluster 340 determines whether to start/continue at block 330 and stop, at block 392, the candidate system simulations 350 (350-1, . . . , 350-N), which may be based on a predetermined parameter. At block 342, the molecular dynamics simulation cluster 340 stores simulation results in a database 360, or other like designated location (e.g., a cloud data warehouse or data lake). In some aspects of the present disclosure, the immediate results of the simulation are collections of coordinates of individual atoms involved in the simulation and their evolution in time.

At block 406, the plurality of candidate systems are re-ranked according to the corresponding properties of interest predicted by a second prediction model. For example, as shown in FIG. 3, the second prediction model 380 can be applied at various stages of progress of the simulation by the molecular dynamics simulation cluster 340. The progress, as well as the interval and frequency at which the second prediction model 380 is applied to re-rank the top-N candidates 322, is a predetermined parameter of the entire system, which can be optimized for best performance to start/continue the molecular dynamics simulation at block 330 and stop the molecular dynamics simulation at block 392. Optionally, further prediction models can be applied as they become available to aid in the prioritization of material candidates at block 390.

At block 408, a second top-N of the plurality of candidate systems is simulated according to the corresponding properties of interest predicted by the second prediction model. For example, as shown in FIG. 3, the second prediction model 380 predicts a resulting lithium (Li+) ion conductivity and/or a simulation process and an estimate of the prediction uncertainty. In this example, the second prediction model 380 performs the predictions from an input of available trajectory data of a material's partially progressed simulation, for example, based on the candidate system simulations 350 (350-1, . . . , 350-N) of the molecular dynamics simulation cluster 340. The spatial-temporal data from the not yet completed simulation trajectory (e.g., a partially completed material candidate simulation) is fed into the second prediction model 380 to obtain the predicted quantities for re-ranking the candidate systems to provide a second one of the top-N candidates 322. In some aspects of the present disclosure, the predicted quantities are used to re-establish rank according to the trajectory process at block 390.

The method 400 includes simulating the second top-N of the plurality of candidate systems by reprioritizing and reassigning computer resources for the simulating based on the re-ranking of the plurality of candidate systems. The method 400 also includes re-training the first prediction model and/or the second prediction model in response to availability of a latest ground-truth data. The method 400 further includes computing a conductivity and a diffusivity of lithium (Li+) ions in the plurality of candidate systems. The method 400 also includes the second prediction model predicting a conductivity and a diffusivity of lithium (Li+) ions in a partially completed material candidate simulation.

The method 400 also includes which ranking by predicting of property estimates for a diffusivity property and a conductivity property of the plurality of candidate systems and an associated measure of prediction uncertainty. The method 400 also includes ranking the plurality of candidate systems according to the predicting of property estimates for the diffusivity property and the conductivity property of the plurality of candidate systems. The method 400 also includes an interval and frequency at which the second prediction model is applied is a predetermined parameter to start/continue a molecular dynamics simulation of the second top-N of the plurality of candidate systems. The method 400 further includes selecting one or more polymers, one or more salts, a concentration of each component, and a temperature to identify a suitable polymer electrolyte material to use in a battery from the second top-N of the plurality of candidate systems, for example, as shown in FIG. 3.

In some aspects, the method 400 may be performed by the SoC 100 (FIG. 1). That is, each of the elements of the method 400 may, for example, but without limitation, be performed by the SoC 100 or one or more processors (e.g., CPU 102 and/or NPU 108) and/or other components included therein.

The various operations of methods described above may be performed by any suitable means capable of performing the corresponding functions. The means may include various hardware and/or software component(s) and/or module(s), including, but not limited to, a circuit, an application specific integrated circuit (ASIC), or processor. Generally, where there are operations illustrated in the figures, those operations may have corresponding counterpart means-plus-function components with similar numbering.

As used, the term “determining” encompasses a wide variety of actions. For example, “determining” may include calculating, computing, processing, deriving, investigating, looking up (e.g., looking up in a table, a database or another data structure), ascertaining and the like. Additionally, “determining” may include receiving (e.g., receiving information), accessing (e.g., accessing data in a memory) and the like. Furthermore, “determining” may include resolving, selecting, choosing, establishing, and the like.

As used, a phrase referring to “at least one of” a list of items refers to any combination of those items, including single members. As an example, “at least one of: a, b, or c” is intended to cover: a, b, c, a-b, a-c, b-c, and a-b-c.

The various illustrative logical blocks, modules and circuits described in connection with the present disclosure may be implemented or performed with a general purpose processor, a digital signal processor (DSP), an application specific integrated circuit (ASIC), a field programmable gate array signal (FPGA) or other programmable logic device (PLD), discrete gate or transistor logic, discrete hardware components or any combination thereof designed to perform the functions described. A general purpose processor may be a microprocessor, but in the alternative, the processor may be any commercially available processor, controller, microcontroller, or state machine. A processor may also be implemented as a combination of computing devices, e.g., a combination of a DSP and a microprocessor, a plurality of microprocessors, one or more microprocessors in conjunction with a DSP core, or any other such configuration.

The steps of a method or algorithm described in connection with the present disclosure may be embodied directly in hardware, in a software module executed by a processor, or in a combination of the two. A software module may reside in any form of storage medium that is known in the art. Some examples of storage media that may be used include random access memory (RAM), read-only memory (ROM), flash memory, erasable programmable read-only memory (EPROM), electrically erasable programmable read-only memory (EEPROM), registers, a hard disk, a removable disk, a CD-ROM, and so forth. A software module may comprise a single instruction, or many instructions, and may be distributed over several different code segments, among different programs, and across multiple storage media. A storage medium may be coupled to a processor such that the processor can read information from, and write information to, the storage medium. In the alternative, the storage medium may be integral to the processor.

The methods disclosed, include one or more steps or actions for achieving the described method. The method steps and/or actions may be interchanged with one another without departing from the scope of the claims. In other words, unless a specific order of steps or actions is specified, the order and/or use of specific steps and/or actions may be modified without departing from the scope of the claims.

The functions described may be implemented in hardware, software, firmware, or any combination thereof. If implemented in hardware, an example hardware configuration may comprise a processing system in a device. The processing system may be implemented with a bus architecture. The bus may include any number of interconnecting buses and bridges depending on the specific application of the processing system and the overall design constraints. The bus may link together various circuits including a processor, machine-readable media, and a bus interface. The bus interface may be used to connect a network adapter, among other things, to the processing system via the bus. The network adapter may be used to implement signal processing functions. For certain aspects, a user interface (e.g., keypad, display, mouse, joystick, etc.) may also be connected to the bus. The bus may also link various other circuits such as timing sources, peripherals, voltage regulators, power management circuits, and the like, which are well known in the art, and therefore, will not be described any further.

The processor may be responsible for managing the bus and general processing, including the execution of software stored on the machine-readable media. The processor may be implemented with one or more general purpose and/or special-purpose processors. Examples include microprocessors, microcontrollers, DSP processors, and other circuitry that can execute software. Software shall be construed broadly to mean instructions, data, or any combination thereof, whether referred to as software, firmware, middleware, microcode, hardware description language, or otherwise. Machine-readable media may include, by way of example, random access memory (RAM), flash memory, read-only memory (ROM), programmable read-only memory (PROM), erasable programmable read-only memory (EPROM), electrically erasable programmable read-only memory (EEPROM), registers, magnetic disks, optical disks, hard drives, or any other suitable storage medium, or any combination thereof. The machine-readable media may be embodied in a computer-program product. The computer-program product may comprise packaging materials.

In a hardware implementation, the machine-readable media may be part of the processing system separate from the processor. However, as those skilled in the art will readily appreciate, the machine-readable media, or any portion thereof, may be external to the processing system. By way of example, the machine-readable media may include a transmission line, a carrier wave modulated by data, and/or a computer product separate from the device, all which may be accessed by the processor through the bus interface. Alternatively, or in addition, the machine-readable media, or any portion thereof, may be integrated into the processor, such as the case may be with cache and/or general register files. Although the various components discussed may be described as having a specific location, such as a local component, they may also be configured in various ways, such as certain components being configured as part of a distributed computing system.

The processing system may be configured as a general purpose processing system with one or more microprocessors providing the processor functionality and external memory providing at least a portion of the machine-readable media, all linked together with other supporting circuitry through an external bus architecture. Alternatively, the processing system may comprise one or more neuromorphic processors for implementing the neuron models and models of neural systems described. As another alternative, the processing system may be implemented with an application specific integrated circuit (ASIC) with the processor, the bus interface, the user interface, supporting circuitry, and at least a portion of the machine-readable media integrated into a single chip, or with one or more field programmable gate arrays (FPGAs), programmable logic devices (PLDs), controllers, state machines, gated logic, discrete hardware components, or any other suitable circuitry, or any combination of circuits that can perform the various functionality described throughout this disclosure. Those skilled in the art will recognize how best to implement the described functionality for the processing system depending on the particular application and the overall design constraints imposed on the overall system.

The machine-readable media may comprise a number of software modules. The software modules include instructions that, when executed by the processor, cause the processing system to perform various functions. The software modules may include a transmission module and a receiving module. Each software module may reside in a single storage device or be distributed across multiple storage devices. By way of example, a software module may be loaded into RAM from a hard drive when a triggering event occurs. During execution of the software module, the processor may load some of the instructions into cache to increase access speed. One or more cache lines may then be loaded into a general register file for execution by the processor. When referring to the functionality of a software module below, it will be understood that such functionality is implemented by the processor when executing instructions from that software module. Furthermore, it should be appreciated that aspects of the present disclosure result in improvements to the functioning of the processor, computer, machine, or other system implementing such aspects.

If implemented in software, the functions may be stored or transmitted over as one or more instructions or code on a non-transitory computer-readable medium. Computer-readable media include both computer storage media and communication media including any medium that facilitates transfer of a computer program from one place to another. A storage medium may be any available medium that can be accessed by a computer. By way of example, and not limitation, such computer-readable media can comprise RAM, ROM, EEPROM, CD-ROM or other optical disk storage, magnetic disk storage, or other magnetic storage devices, or any other medium that can be used to carry or store desired program code in the form of instructions or data structures and that can be accessed by a computer. Additionally, any connection is properly termed a non-transitory computer-readable medium. For example, if the software is transmitted from a website, server, or other remote source using a coaxial cable, fiber optic cable, twisted pair, digital subscriber line (DSL), or wireless technologies such as infrared (IR), radio, and microwave, then the coaxial cable, fiber optic cable, twisted pair, DSL, or wireless technologies such as infrared, radio, and microwave are included in the definition of medium. Disk and disc, as used herein, include compact disc (CD), laser disc, optical disc, digital versatile disc (DVD), floppy disk, and Blu-ray® disc, where disks usually reproduce data magnetically, while discs reproduce data optically with lasers. Thus, in some aspects computer-readable media may comprise non-transitory computer-readable media (e.g., tangible media). In addition, for other aspects computer-readable media may comprise transitory computer-readable media (e.g., a signal). Combinations of the above should also be included within the scope of computer-readable media.

Thus, certain aspects may comprise a computer program product for performing the operations presented. For example, such a computer program product may comprise a non-transitory computer-readable medium having instructions stored (and/or encoded) thereon, the instructions being executable by one or more processors to perform the operations described herein. For certain aspects, the computer program product may include packaging material.

Further, it should be appreciated that modules and/or other appropriate means for performing the methods and techniques described herein, may be downloaded and/or otherwise obtained by a user terminal and/or base station as applicable. For example, such a device can be coupled to a server to facilitate the transfer of means for performing the methods described herein. Alternatively, various methods described herein, may be provided via storage means (e.g., RAM, ROM, a physical storage medium such as a compact disc (CD) or floppy disk, etc.), such that a user terminal and/or base station can obtain the various methods upon coupling or providing the storage means to the device. Moreover, any other suitable technique for providing the methods and techniques described herein to a device can be utilized.

It is to be understood that the claims are not limited to the precise configuration and components illustrated above. Various modifications, changes, and variations may be made in the arrangement, operation, and details of the methods and apparatus described above without departing from the scope of the claims.

What is claimed is:

1. A method of closed loop simulation for accelerated material discovery, comprising:
ranking a plurality of candidate systems according to corresponding properties of interest predicted by a first prediction model;
simulating a first top-N of the plurality of candidate systems according to the corresponding properties of interest predicted by the first prediction model;
re-ranking the plurality of candidate systems according to the corresponding properties of interest predicted by a second prediction model; and
simulating a second top-N of the plurality of candidate systems according to the corresponding properties of interest predicted by the second prediction model.

2. The method of claim 1, in which simulating the second top-N of the plurality of candidate systems comprises reprioritizing and reassigning computer resources for the simulating based on the re-ranking of the plurality of candidate systems.

3. The method of claim 1, further comprising re-training the first prediction model and/or the second prediction model in response to availability of a latest ground-truth data.

4. The method of claim 1, further comprising computing a conductivity and a diffusivity of lithium (Li+) ions in the plurality of candidate systems.

5. The method of claim 4, in which ranking comprises:
predicting of property estimates for a diffusivity property and a conductivity property of the plurality of candidate systems and an associated measure of prediction uncertainty; and
ranking the plurality of candidate systems according to the predicting of property estimates for the diffusivity property and the conductivity property of the plurality of candidate systems.

6. The method of claim 1, in which the second prediction model predicts a conductivity and a diffusivity of lithium (Li+) ions in a partially completed material candidate simulation.

7. The method of claim 1, in which an interval and frequency at which the second prediction model is applied is a predetermined parameter to start/continue a molecular dynamics simulation of the second top-N of the plurality of candidate systems.

8. The method of claim 1, further comprises selecting one or more polymers, one or more salts, a concentration of each component, and a temperature to identify a suitable polymer electrolyte material to use in a battery from the second top-N of the plurality of candidate systems.

9. A non-transitory computer-readable medium having program code recorded thereon for compositional feature representation learning of closed loop simulation for accelerated material discovery, the program code being executed by a processor and comprising:
program code to rank a plurality of candidate systems according to corresponding properties of interest predicted by a first prediction model;
program code to simulate a first top-N of the plurality of candidate systems according to the corresponding properties of interest predicted by the first prediction model;
program code to re-rank the plurality of candidate systems according to the corresponding properties of interest predicted by a second prediction model; and
program code to simulate a second top-N of the plurality of candidate systems according to the corresponding properties of interest predicted by the second prediction model.

10. The non-transitory computer-readable medium of claim 9, in which the program code to simulate the second top-N of the plurality of candidate systems comprises program code to reprioritize and reassigning computer resources for the simulating based on the re-rank of the plurality of candidate systems.

11. The non-transitory computer-readable medium of claim 9, further comprising program code to re-train the first prediction model and/or the second prediction model in response to availability of a latest ground-truth data.

12. The non-transitory computer-readable medium of claim 9, further comprising program code to compute a conductivity and a diffusivity of lithium (Li+) ions in the plurality of candidate systems.

13. The non-transitory computer-readable medium of claim 12, in which the program code to rank comprises:
program code to predict of property estimates for a diffusivity property and a conductivity property of the plurality of candidate systems and an associated measure of prediction uncertainty; and
program code to rank the plurality of candidate systems according to the predicting of property estimates for the diffusivity property and the conductivity property of the plurality of candidate systems.

14. The non-transitory computer-readable medium of claim 9, in which the second prediction model predicts a conductivity and a diffusivity of lithium (Li+) ions in a partially completed material candidate simulation.

15. The non-transitory computer-readable medium of claim 9, in which an interval and frequency at which the second prediction model is applied is a predetermined parameter to start/continue a molecular dynamics simulation of the second top-N of the plurality of candidate systems.

16. The non-transitory computer-readable medium of claim 9, further comprises program code to select one or more polymers, one or more salts, a concentration of each component, and a temperature to identify a suitable polymer electrolyte material to use in a battery from the second top-N of the plurality of candidate systems.

17. A system for compositional feature representation learning of closed loop simulation for accelerated material discovery, the system comprising:
a first prediction model to rank a plurality of candidate systems according to corresponding properties of interest predicted by the first prediction model;
a molecular dynamics simulation cluster to simulate a first top-N of the plurality of candidate systems according to the corresponding properties of interest predicted by the first prediction model;
a second prediction model to re-rank the plurality of candidate systems according to the corresponding properties of interest predicted by the second prediction model; and
the molecular dynamics simulation cluster to simulate a second top-N of the plurality of candidate systems according to the corresponding properties of interest predicted by the second prediction model.

18. The system of claim 17, in which the molecular dynamics simulation cluster is further to reprioritize and reassign computer resources for the simulating based on the re-rank of the plurality of candidate systems.

19. The system of claim 17, in which the first prediction model and/or the second prediction model to re-train in response to availability of a latest ground-truth data.

20. The system claim 17, in which the second prediction model is further to predict of property estimates for a diffusivity property and a conductivity property of the plurality of candidate systems and an associated measure of prediction uncertainty, and to rank the plurality of candidate systems according to the predicting of property estimates for the diffusivity property and the conductivity property of the plurality of candidate systems.
